(19) United States
(12) Patent Application Publication
(10) Pub. No.: US 2023/0176021 A1
(43) Pub. Date: Jun. 8, 2023

(54) AGRICULTURAL MULTIFUNCTIONAL CARBON DIOXIDE CONTROLLER

(71) Applicant: KUNSHAN REYING PHOTOELECTRICITY CO., LTD., Suzhou (CN)

(72) Inventors: Yuchen HE, Suzhou (CN); Yonggang ZANG, Suzhou (CN); Yuanfeng WANG, Suzhou (CN); Weibing WANG, Suzhou (CN); Jing ZHAO, Suzhou (CN); Lin LI, Suzhou (CN)

(21) Appl. No.: 17/926,008

(22) PCT Filed: May 14, 2021
(86) PCT No.: PCT/CN2021/093810

§ 371 Date: Nov. 17, 2022

(30) Foreign Application Priority Data
May 20, 2020 (CN) 202010432252.7

(51) Int. Cl. G01N 33/00; G08B 21/14

(52) U.S. Cl. CPC G01N 33/0062; G08B 21/14; G01N 33/0063

(57) ABSTRACT

An agricultural multifunctional carbon dioxide controller, comprising a master machine which is capable of carrying out Bluetooth signal transmission with an external display. A slave machine and a power plug are connected to the master machine, the master machine is connected to the slave machine by means of a network line, and the master machine is connected to the power plug by means of a power line; a connectable fan, a power supply system or another external device are connected to the power line in parallel; and the master machine can read data measured by the slave machine in real time and can control the fan, the power supply system and the slave machine.

### CROSS-REFERENCE TO RELATED APPLICATION

This patent application claims the benefit and priority of Chinese Patent Application No. 202010432252.7, filed with the China National Intellectual Property Administration on May 20, 2020, the disclosure of which is incorporated by reference herein in its entirety as part of the present application.

### TECHNICAL FIELD

The present disclosure relates to the field of carbon dioxide monitoring, and in particular relates to an agricultural multifunctional carbon dioxide controller.

### BACKGROUND

The carbon dioxide detector can effectively measure the content of carbon dioxide in indoor air, and is mainly applied to detecting the concentration of carbon dioxide in industrial, agricultural and living environments.

However, existing agricultural multifunctional carbon dioxide controller can only monitor the carbon oxide, temperature and humidity in real time, and the display interface is mostly an ordinary LCD, thus the user cannot inquire about the past historical information, the display content is limited, the limitation is relatively large, and the operation is inflexible.

### SUMMARY

The technical problem to be solved by the present disclosure is to provide an agricultural multifunctional carbon dioxide controller which can be conveniently used for inquiring about historical monitoring data.

To solve the technical problem above, the present disclosure is achieved through the following technical solution: An agricultural multifunctional carbon dioxide controller comprises a master machine which is capable of carrying out Bluetooth signal transmission with an external display. A slave machine and a power plug are connected to the master machine, the master machine is connected to the slave machine by means of a network line, and the master machine is connected to the power plug by means of a power line. A fan, a power supply system or another external device are connected to the power line in parallel; and the master machine can read data measured by the slave machine in real time and can control the fan, the power supply system and the slave machine.

Preferably, the master machine is composed of a master machine rear cover, a master machine circuit board and a master machine front cover. The front face of the master machine rear cover is provided with a first cavity with an outward opening, the master machine circuit board is fixed into the first cavity, and the master machine front cover is fixed to the front face of the master machine rear cover by means of bolts and seals the master machine circuit board. The front face of the master machine front cover is provided with a master machine panel. A master machine buzzer is welded to the upper right of the front face of the master machine circuit board, and a dot-matrix liquid crystal display screen is welded to the middle of the master machine circuit board. Two buffer pads arranged in parallel are arranged between the dot-matrix liquid crystal display screen and the master machine circuit board, and the dot-matrix liquid crystal display screen is plugged into a socket welded to the left of center on the front face of the master machine circuit board. Four independent master machine LED (light-emitting diode) lamps, three independent control buttons and one master machine reset button are welded to the portion, below the dot-matrix liquid crystal display screen, on the mater machine circuit board, and the control buttons and the master machine reset button are located directly below the master machine LED lamps. A Bluetooth module is welded to the lower left of the front face of the master machine circuit board, two memory chips and a relay are welded to the back face of the master machine circuit board, and the memory chips are located above the relay. A button battery holder, a wiring terminal, a power module, a safety capacitor and a common mode choke are also welded to the back face of the master machine circuit board. The bottom of the master machine rear cover is provided with a plurality of wiring holes communicating with the first cavity in the master machine rear cover, the wiring terminals on the master machine circuit board correspond to the wiring holes. Waterproof cable fixing heads are sleeved outside the wiring holes. The external network line and the power line are plugged into the wiring terminals after penetrating through the cable fixing heads.

Preferably, the master machine front cover is provided with a display hole capable of being clamped with the dot-matrix liquid crystal display screen on the master machine circuit board and lamp holes corresponding to the four master machine LED lamps on the master machine circuit board in number and position. The heads of the master machine LED lamps are plugged into the corresponding lamp holes. The master machine front cover and the master machine panel are both provided with button holes, the number of the button holes is equal to that of the control buttons and the master reset button on the master circuit board, and the button holes correspond to the control buttons and the master reset button in position. Master machine buttons made of rubber are arranged in the button holes, and the tail ends of the master machine buttons are in contact with the corresponding control buttons and the master reset button.

Preferably, a first waterproof ring is arranged at the joint of the master machine rear cover and the master machine front cover, and a fixing frame is arranged at the back face of the master machine rear cover.

Preferably, the slave machine is composed of a slave machine rear cover, a slave machine front cover and a slave machine circuit board. The back face of the slave machine front cover is provided with an accommodating cavity with an outward opening, the slave machine circuit board is fixed into the accommodating cavity, and the slave machine rear cover is fixed to the back face of the slave machine front cover by means of bolts, thus sealing the slave machine circuit board. The slave machine circuit board is provided with a gap near the upper part, a carbon dioxide sensor is arranged in the gap, and the carbon dioxide sensor is welded to the slave machine circuit board. The middle portion of the front face of the slave machine circuit board is provided with a slave machine liquid crystal display screen. The slave machine liquid crystal display screen is connected to the slave machine circuit board by two conductive rubbers vertically arranged in parallel, and buffer pads are arranged between the conductive rubbers and the slave machine liquid crystal display screen. Two slave machine LED lights are welded to the portion, below the slave machine liquid crystal display screen, on the front face of the slave machine circuit board. A network line for connecting the master machine is welded below the slave machine circuit board. A sensor small plate is arranged directly below the slave machine LED lamp, the sensor small plate is connected to a communication line socket welded to the slave machine circuit board, a temperature-humidity sensor, a pressure sensor and a photosensitive sensor are welded to the front face of the sensor small plate.

Preferably, a portion, corresponding to the carbon dioxide sensor, on the slave machine rear cover is provided with a front-back through hollow-out portion. The front face of the slave machine front cover is sequentially provided with an air vent, a display window, lamp holes and a connector communicating with the back face of the slave machine front cover from top to bottom. The air vent, the display window, the lamp holes and the connector correspond to the carbon dioxide sensor, the slave machine liquid crystal display screen, the slave machine LED lamps and the sensor small plate on the slave machine circuit board, respectively. A slave machine panel for sealing and covering the air vent, the display window, the lamp holes and the connector is attached to the exterior of the front face of the slave machine front cover, and the slave machine panel is provided with two air inlet holes corresponding to the air vent and the connector, respectively.

Preferably, waterproof breathable membranes are arranged between the sensor small plate and the air inlet hole in the slave machine panel and between the carbon dioxide sensor and the hollowed-out portion in the slave machine rear cover.

Preferably, the slave machine front cover is provided with a lead hole at the position corresponding to the portion where the network line is welded to the slave machine circuit board, and the network line is welded to the slave machine circuit board after penetrating through the lead hole.

Preferably, the top of the slave machine rear cover protrudes upwards, and a hanging hole is formed in the protruded portion. A slave machine waterproof ring is arranged at a position where the slave machine front cover is in fit with the slave machine rear cover.

Preferably, the display is a computer or a mobile phone. The power supply system is a power supply socket.

Compared with the prior art, the present disclosure has the beneficial effects that the agricultural multifunctional carbon dioxide controller can analyze the effect of weather changes and sunshine duration on crop production, a user can directly monitor related data, and a client can read monitored data conveniently; at the same time, the client can inquire about historical monitored data and can also select different modes to adapt to different types of crop production; the master machine meets production requirements according to specific environments; and the agricultural multifunctional carbon dioxide controller is high in practicability, flexible in use and convenient to popularize.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a structure diagram of an agricultural multifunctional carbon dioxide controller in accordance with the present disclosure;

FIG. 2 is a schematic diagram of a master machine split structure in an agricultural multifunctional carbon dioxide controller in accordance with the present disclosure;

FIG. 3 is a schematic diagram of a slave machine split structure in an agricultural multifunctional carbon dioxide controller in accordance with the present disclosure.

In the drawings: 1—master machine; 11—master machine rear cover; 111—first cavity; 112—wiring hole; 12—master machine front cover; 121—display hole; 13—master machine panel; 131—button hole; 14—master machine circuit board; 140—dot-matrix liquid crystal display screen; 1401—socket; 1402—buffer pad; 141—wiring terminal; 142—power module; 143—safety capacitor; 144—common mode choke; 145—Bluetooth module; 146—memory chip; 147—relay; 148—master machine buzzer; 149—master machine LED lamp; 15—first waterproof ring; 16—master machine button; 161—control button; 162—master machine reset button; 17—fixing frame; 2—slave machine; 21—slave machine rear cover; 211—hollow-out portion; 22—slave machine front cover; 221—air vent; 222—display window; 223—connector; 23—slave machine panel; 231—air inlet hole; 24—slave machine circuit board; 241—carbon dioxide sensor; 242—communication line socket; 243—slave machine liquid crystal display screen; 2431—conductive rubber; 2432—buffer pad; 244—sensor small plate; 245—temperature-humidity sensor; 246—pressure sensor; 247—photosensitive sensor; 248—slave machine LED lamp; 25—waterproof breathable membrane; 26—salve machine waterproof ring; 3—display; 4—network line; 5—power line; 6—power plug; 7—fan; 8—power supply system.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

To make the objectives, features and advantages of the present disclosure more apparently and understandably, the present disclosure is further described in detail below with reference to the accompanying drawings and specific embodiments.

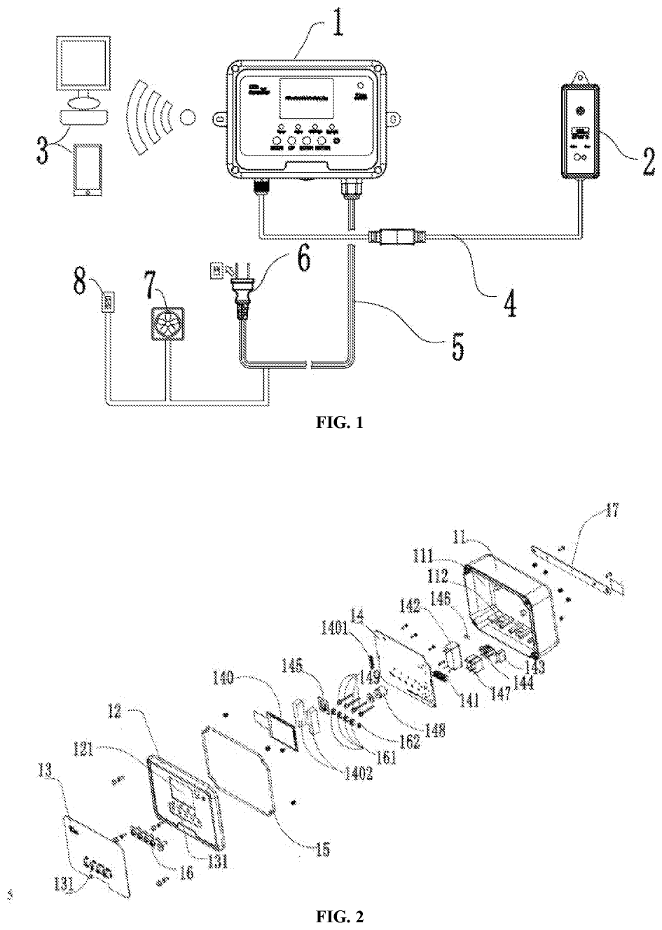

As shown in FIG. 1, an agricultural multifunctional carbon dioxide controller comprises a master machine 1 which is capable of carrying out Bluetooth signal transmission with an external display 3. The display 3 is a computer or a mobile phone. A slave machine 2 and a power plug 6 are connected to the master machine 1. The master machine 1 is connected to the slave machine 2 by means of a network line 4, and the master machine 1 is connected to the power plug 6 by means of a power line 5. A fan 7, a power supply system 8 or another external device are connected to the power line 5 in parallel, and the power supply system 8 is a power supply socket to facilitate the plugging and power-on of other external devices such as air-conditioners. The master machine 1 can read data measured by the slave machine 2 in real time and can control the fan 7, the power supply system 8 and the slave machine 2.

As shown in FIG. 2, the master machine 1 is composed of a master machine rear cover 11, a master machine circuit board 14 and a master machine front cover 12. The front face of the master machine rear cover 11 is provided with a first cavity 111 with an outward opening, the master machine circuit board 14 is fixed into the first cavity 111, the master machine front cover 12 is fixed to the front face of the master machine rear cover 11 and seals the master machine circuit board 14. The front face of the master machine front cover 12 is provided with a master machine panel 13. A master machine buzzer 148 is welded to the upper right of the front face of the master machine circuit board 14, and may give an alarm when the carbon dioxide concentration, the temperature and the humidity exceed preset values or preset range of a user. A dot-matrix liquid crystal display screen 140 is welded to the middle of the front face of the master machine circuit board 14. The dot-matrix liquid crystal display screen 140 is a color screen, and the design of the color screen is an upgrade of an original product, through which the user can read monitored values better in a chart display manner, and a client can analyze data through the mean value, the maximum value or the minimum value. Two buffer pads 1402 arranged in parallel are arranged between the dot-matrix liquid crystal display screen 140 and the master machine circuit board 14, and the dot-matrix liquid crystal display screen 140 is plugged into a socket 1401 welded to the left of center on the front face of the master machine circuit board 14. Four independent master machine LED lamps 149, three independent control buttons 161 and one master machine reset button 162 are welded to the portion, below the dot-matrix liquid crystal display screen 140, on the mater machine circuit board 14, and related control program is upgraded such that the user can call out the historical data of a certain period of time and select different display and control modes to meet the production requirements of different crops. The control buttons 161 and the master machine reset button 162 are located directly below the master machine LED lamps 149. The four independent master machine LED lamps respectively denote whether the controller is connected to the power supply or not, whether a relay is turned on or not, whether the carbon dioxide exceeds the standard or not, and whether the lighting is effective or not, such that the user can master basic conditions of an environment where the instrument is located. A Bluetooth module 145 is welded to the lower left of the front face of the master machine circuit board 14, such that the user not only can read and analyze data on the instrument, but also can transmit the data to the display 3, such as a computer or a mobile phone, for data analysis, storage or sharing. Two memory chips 146 and the relay 147 are welded to the back face of the master machine circuit board 14, and the fan 7, the power supply system or other external device connected to the master machine 1 in parallel are controlled by the relay 147, the memory chips 146 are located above the relay 147, and are configured to save historic data monitored by the instrument. When the carbon dioxide, temperature and humidity or lighting duration is not within a range set by a user, the relay 147 can control the external devices such as a carbon gas cylinder, a fan and a lighting lamp to adjust the environment in the greenhouse so as to meet production requirements. A button battery holder, a wiring terminal 141, a power module 142, a safety capacitor 143 and a common mode choke 144 are also welded to the back face of the master machine circuit board 14, and the button battery holder enables time setting not to disappear when the master machine 1 is in a power-off state. The bottom of the master machine rear cover 11 is provided with a plurality of wiring holes 112 communicating with the first cavity 111 in the master machine rear cover, and the wiring terminals 141 on the master machine circuit board 14 correspond to the wiring holes 112. The external network line 4 and the power line 5 are plugged into the wiring terminals 141 after penetrating through the cable fixing heads.

To facilitate the master machine circuit board 14 to be in fit with the master machine front cover 12, the master machine front cover 12 is provided with a display hole 121 capable of being clamped with the dot-matrix liquid crystal display screen 140 on the master machine circuit board 14 and lamp holes corresponding to the four master machine LED lamps 149 on the master machine circuit board 14 in number and position. The heads of the master machine LED lamps 149 are plugged into the corresponding lamp holes. The master machine front cover 12 and the master machine panel 13 are both provided with button holes 131, the number of the button holes 131 is equal to that of the control buttons 161 and the master reset button 162 on the master circuit board 14, and the button holes 131 correspond to the control buttons 161 and the master reset button 162 in position. Master machine buttons 16 made of rubber are arranged in the button holes 131, and the tail ends of the master machine buttons 16 are in contact with the corresponding control buttons 161 and the master reset button 162.

To prevent the water from entering the master machine 1 and affecting the components on the master machine circuit board 14, a first waterproof ring 15 is arranged at the joint of the master machine rear cover 11 and the master machine front cover 12. Waterproof cable fixing heads are sleeved outside the wiring holes 112; the external network line 4 and the power line 5 are plugged into the wiring terminals 141_L after penetrating through the cable fixing heads. To facilitate the installation of the master machine 1, the back face of the master machine rear cover 11 is provided with a fixing frame 17.

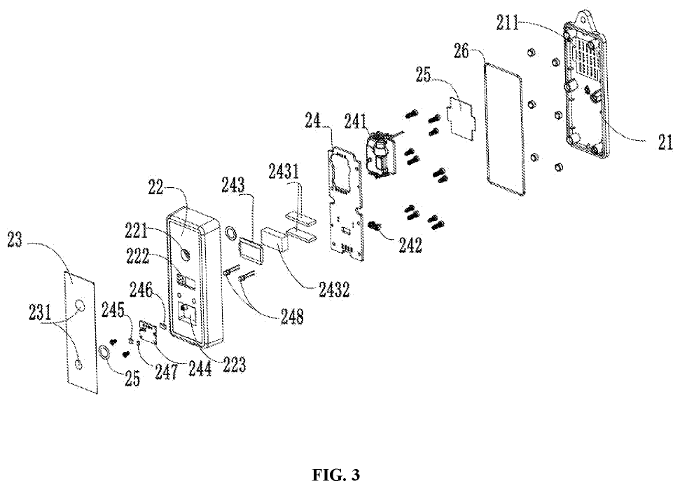

As shown in FIG. 3, the slave machine 2 is composed of a slave machine rear cover 21, a slave machine front cover 22, and a slave machine circuit board 24. The back face of the slave machine front cover 22 is provided with an accommodating cavity with an outward opening, the slave machine circuit board 24 is fixed into the accommodating cavity, and the slave machine rear cover 21 is fixed to the back face of the slave machine front cover 22 by means of bolts, thus sealing the slave machine circuit board 24. The slave machine circuit board 24 is provided with a gap near the upper part, a carbon dioxide sensor 241 is arranged in the gap, the carbon dioxide sensor 241 is welded to the slave machine circuit board 24, and the carbon dioxide sensor 241 is configured to monitor the concentration of carbon dioxide in the environment. The middle portion of the front face of the slave machine circuit board 24 is provided with a slave machine liquid crystal display screen 243 which is configured to circularly display the numerical values of the current carbon dioxide, the temperature and humidity. The slave machine liquid crystal display screen 243 is connected to the slave machine circuit board 24 by two conductive rubbers 2431 vertically arranged in parallel, and buffer pads 2432 are arranged between the conductive rubbers 2431 and the slave machine liquid crystal display screen 243. Two slave machine LED lights 248 are welded to the portion, below the slave machine liquid crystal display screen 243, on the front face of the slave machine circuit board 24 to respectively display whether the relay is turned on or not and whether the current lighting is effective or not. A network line 4 for connecting the master machine 1 is welded below the slave machine circuit board 24. A sensor small plate 244 is arranged directly below the slave machine LED lamps 248, and the sensor small plate 244 is connected to a communication line socket 242 welded to the slave machine circuit board 24. A temperature-humidity sensor 245, a pressure sensor 246 and a photosensitive sensor 247 are welded to the front face of the sensor small plate 244 and configured to monitor air pressure and lighting intensity, such that the user can analyze the effect of weather changes and sunshine duration on crop production.

To facilitate the ventilation of the slave machine and to ensure the operation of the carbon dioxide sensor 241 and other components, a portion, corresponding to the carbon dioxide sensor 241, on the slave machine rear cover 21 is provided with a front-back through hollow-out portion 211. The front face of the slave machine front cover 22 is sequentially provided with an air vent 221, a display window 222, lamp holes and a connector 223 communicating with the back face of the slave machine front cover 22 from top to bottom. The air vent 221, the display window 222, the lamp holes and the connector 223 correspond to the carbon dioxide sensor 241, the slave machine liquid crystal display screen 243, the slave machine LED lamps 248 and the sensor small plate 244 on the slave machine circuit board 24, respectively. A slave machine panel 23 for sealing and covering the air vent 221, the display window 222, the lamp holes and the connector 223 is attached to the exterior of the front face of the slave machine front cover 22. The slave machine panel 23 is provided with two air inlet holes 231 corresponding to the air vent 221 and the connector 223, respectively.

To prevent the water from entering the slave machine 2, waterproof breathable membranes 25 are arranged between the sensor small plate 244 and the air inlet hole 231 on the slave machine panel 23 and between the carbon dioxide sensor 241 and the hollow-out portion 211 in the slave machine rear cover 21. A slave machine waterproof ring 26 is provided at the position where the slave machine front cover 22 is in fit with the slave machine rear cover 21.

To facilitate the network line 4 to be connected to the slave machine circuit board 24 after penetrating through the slave machine front cover 2, the slave machine front cover 22 is provided with a lead hole at the position corresponding to the portion where the network line 4 is welded to the slave machine circuit board 24, and the network line 4 is welded to the slave machine circuit board 24 after penetrating through the lead hole.

To facilitate the installation of the slave machine 2, the top of the slave machine rear cover 21 protrudes upwards, and a hanging hole is formed in the protruded portion.

The specific operation principle of the controller is as follows: the master machine 1 and the slave machine 2 are installed according to specific environmental requirements. The slave machine 2 is configured to monitor the carbon dioxide concentration, the temperature and humidity, pressure and lighting duration in the environment by means of the carbon dioxide sensor 241, the temperature and humidity sensor 245, the pressure sensor 246 and the photosensitive sensor 247 in the slave machine 2, and monitored data is displayed and integrated by the master machine 1 connected to the slave machine 2 by means of the network line 4, thus facilitating the user to read the data. Data displayed on the master machine 1 can be transmitted to a computer or a mobile phone of the user by means of the Bluetooth module 145 in the master machine 1 for data analysis, storage or sharing. Moreover, when the carbon dioxide, temperature and humidity or lighting duration in the agricultural greenhouse is not within the range set by the user, the master machine can control external devices such as a carbon dioxide gas cylinder, a fan and a lighting lamp to adjust the environment in the greenhouse to meet the production requirements by means of the relay.

The agricultural multifunctional carbon dioxide controller can analyze the effect of weather changes and sunshine duration on crop production, a user can directly monitor related data, and a client can read monitored data conveniently; at the same time, the client can inquire about historical monitored data and can also select different modes to adapt to different types of crop production; the master machine 1 meets production requirements according to specific environments; and the agricultural multifunctional carbon dioxide controller is high in practicability, flexible in use and convenient to popularize.

It is emphasized that the above is only a preferred embodiment of the present disclosure and is not intended to limit the present disclosure in any form, and any simple modification, equivalent change and modification made on the above embodiment according to the technical essence of the present disclosure still fall within the scope of the technical solution of the present disclosure.

What is claimed is:

1. An agricultural multifunctional carbon dioxide controller, comprising a master machine (1) which is capable of carrying out Bluetooth signal transmission with an external display (3); a slave machine (2) and a power plug (6) are connected to the master machine (1), the master machine (1) is connected to the slave machine (2) by means of a network line (4), and the master machine (1) is connected to the power plug (6) by means of a power line (5); a fan (7), a power supply system (8) or another external device are connected to the power line (5) in parallel; and the master machine (1) is able to read data measured by the slave machine (2) in real time and is able to control the fan (7), the power supply system (8) and the slave machine (2).

2. The agricultural multifunctional carbon dioxide controller according to claim 1, wherein the master machine (1) is composed of a master machine rear cover (11), a master machine circuit board (14) and a master machine front cover (12); the front face of the master machine rear cover (11) is provided with a first cavity (111) with an outward opening, the master machine circuit board (14) is fixed into the first cavity (111), and the master machine front cover (12) is fixed to the front face of the master machine rear cover (11) by means of bolts and seals the master machine circuit board (14); the front face of the master machine front cover (12) is provided with a master machine panel (13); a master machine buzzer (148) is welded to the upper right of the front face of the master machine circuit board (14), and a dot-matrix liquid crystal display screen (140) is welded to the middle of the master machine circuit board; two buffer pads (1402) arranged in parallel are arranged between the dot-matrix liquid crystal display screen (140) and the master machine circuit board (14), and the dot-matrix liquid crystal display screen (140) is plugged into a socket (1401) welded to the left of center on the front face of the master machine circuit board (14); four independent master machine LED lamps (149), three independent control buttons (161) and one master machine reset button (162) are welded to the portion, below the dot-matrix liquid crystal display screen (140), on the mater machine circuit board (14), and the control buttons (161) and the master machine reset button (162) are located directly below the master machine LED lamps (149); a Bluetooth module (145) is welded to the lower left of the front face of the master machine circuit board (14), two memory chips (146) and a relay (147) are welded to the back face of the master machine circuit board (14), and the memory chips (146) are located above the relay (147); a button battery holder, a wiring terminal (141), a power module (142), a safety capacitor (143) and a common mode choke (144) are also welded to the back face of the master machine circuit board (14); the bottom of the master machine rear cover (11) is provided with a plurality of wiring holes (112) communicating with the first cavity (111) in the master machine rear cover, the wiring terminals (141) on the master machine circuit board (14) correspond to the wiring holes (112); waterproof cable fixing heads are sleeved outside the wiring holes (112); the external network line (4) and the power line (5) are plugged into the wiring terminals (141) after penetrating through the cable fixing heads.

3. The agricultural multifunctional carbon dioxide controller according to claim 2, wherein the master machine front cover (12) is provided with a display hole (121) capable of being clamped with the dot-matrix liquid crystal display screen (140) on the master machine circuit board (14) and lamp holes corresponding to the four master machine LED lamps (149) on the master machine circuit board (14) in number and position; the heads of the master machine LED lamps (149) are plugged into the corresponding lamp holes; the master machine front cover (12) and the master machine panel (13) are both provided with button holes (131), the number of the button holes (131) is equal to that of the control buttons (161) and the master reset button (162) on the master circuit board (14), and the button holes (131) correspond to the control buttons (161) and the master reset button (162) in position; and master machine buttons (16) made of rubber are arranged in the button holes (131), and the tail ends of the master machine buttons (16) are in contact with the corresponding control buttons (161) and the master reset button (162).

4. The agricultural multifunctional carbon dioxide controller according to claim 2, wherein a first waterproof ring (15) is arranged at the joint of the master machine rear cover (11) and the master machine front cover (12), and a fixing frame (17) is arranged at the back face of the master machine rear cover (11).

5. The agricultural multifunctional carbon dioxide controller according to claim 1, wherein the slave machine (2) is composed of a slave machine rear cover (21), a slave machine front cover (22) and a slave machine circuit board (24); the back face of the slave machine front cover (22) is provided with an accommodating cavity with an outward opening, the slave machine circuit board (24) is fixed into the accommodating cavity, and the slave machine rear cover (21) is fixed to the back face of the slave machine front cover (22) by means of bolts, thus sealing the slave machine circuit board (24); the slave machine circuit board (24) is provided with a gap near the upper part, a carbon dioxide sensor (241) is arranged in the gap, and the carbon dioxide sensor (241) is welded to the slave machine circuit board (24); the middle portion of the front face of the slave machine circuit board (24) is provided with a slave machine liquid crystal display screen (243); the slave machine liquid crystal display screen (243) is connected to the slave machine circuit board (24) by two conductive rubbers (2431) vertically arranged in parallel, and buffer pads (2432) are arranged between the conductive rubbers (2431) and the slave machine liquid crystal display screen (243); two slave machine LED lights (248) are welded to the portion, below the slave machine liquid crystal display screen (243), on the front face of the slave machine circuit board (24); a network line (4) for connecting the master machine (1) is welded below the slave machine circuit board (24); a sensor small plate (244) is arranged directly below the slave machine LED lamp (248), the sensor small plate (244) is connected to a communication line socket (242) welded to the slave machine circuit board (24), a temperature-humidity sensor (245), a pressure sensor (246) and a photosensitive sensor (247) are welded to the front face of the sensor small plate (244).

6. The agricultural multifunctional carbon dioxide controller according to claim 5, wherein a portion, corresponding to the carbon dioxide sensor (241), on the slave machine rear cover (21) is provided with a front-back through hollow-out portion (211); the front face of the slave machine front cover (22) is sequentially provided with an air vent (221), a display window (222), lamp holes and a connector (223) communicating with the back face of the slave machine front cover (22) from top to bottom; the air vent (221), the display window (222), the lamp holes and the connector (223) correspond to the carbon dioxide sensor (241), the slave machine liquid crystal display screen (243), the slave machine LED lamps (248) and the sensor small plate (244) on the slave machine circuit board (24), respectively; a slave machine panel (23) for sealing and covering the air vent (221), the display window (222), the lamp holes and the connector (223) is attached to the exterior of the front face of the slave machine front cover (22), and the slave machine panel (23) is provided with two air inlet holes (231) corresponding to the air vent (221) and the connector (223), respectively.

7. The agricultural multifunctional carbon dioxide controller according to claim 6, wherein waterproof breathable membranes (25) are arranged between the sensor small plate (244) and the air inlet hole (231) on the slave machine panel (23) and between the carbon dioxide sensor (241) and the hollow-out portion (211) in the slave machine rear cover (21).

8. The agricultural multifunctional carbon dioxide controller according to claim 5, wherein the slave machine front cover (22) is provided with a lead hole at the position corresponding to the portion where the network line (4) is welded to the slave machine circuit board (24), and the network line (4) is welded to the slave machine circuit board (24) after penetrating through the lead hole.

9. The agricultural multifunctional carbon dioxide controller according to claim 5, wherein the top of the slave machine rear cover (21) protrudes upwards, and a hanging hole is formed in the protruded portion; and a slave machine waterproof ring (26) is arranged at a position where the slave machine front cover (22) is in fit with the slave machine rear cover (21).

10. The agricultural multifunctional carbon dioxide controller according to claim 1, wherein the display (3) is a computer or a mobile phone; and the power supply system (6) is a power supply socket.
